(19) United States
(12) Patent Application Publication
(10) Pub. No.: US 2022/0133625 A1
(43) Pub. Date: May 5, 2022

(54) METHOD FOR ELIMINATING SPECIFIC PATHOGENIC MICROORGANISMS IN THE BODY

(71) Applicant: Joachim Hochreuter, Brunnenthal (AT)

(72) Inventor: Joachim Hochreuter, Brunnenthal (AT)

(21) Appl. No.: 17/182,664

(22) Filed: Feb. 23, 2021

Related U.S. Application Data
(63) Continuation-in-part of application No. 16/926,920, filed on Jul. 13, 2020, now abandoned.

(51) Int. Cl. A61K 9/00; A61K 33/00; A61K 31/355; A61K 31/375; A61K 31/198; A61K 31/28; A61K 31/315; A61K 31/232; A61K 31/122; A61K 31/593; A61K 41/00

(52) U.S. Cl. CPC A61K 9/0009; A61K 33/00; A61K 31/355; A61K 31/375; A61K 31/198; A61K 41/0028; A61K 31/315; A61K 31/232; A61K 31/122; A61K 31/593; A61K 9/0053; A61K 31/28

(57) ABSTRACT

The method for eliminating microorganisms, allows to selectively neutralize pathogens. The method includes a first step which includes measuring the frequencies of pathogens within a human body which are at high population present, resulting in further illnesses or symptoms. The different sized pathogens are then applied a resonant frequency band to pre-damage/crack/weaken the hulls of the microorganisms evenly. The method further includes a second step rupturing the damaged hulls of the pathogens or oxidation of the ARN/ADN in case of a virus with a safe dose of an external oxidation product together with internal produced eNOS-expression (ROS/RNS). The external product may include chlorine dioxide in a watery solution of 0.75 ppm, vitamins, minerals and amino acids to start eNOS-expression. The method further includes a third step to deliberately terminating the activated oxidation processes. A fourth step strengthens the immune system by filling depleted levels of vitamins, minerals and amino acids.

### OTHER RELATED APPLICATIONS

The present application is a continuation-in-part of pending U.S. patent application Ser. No. 16/926,920, filed on Jul. 13, 2020, which is hereby incorporated by reference.

### BACKGROUND OF THE INVENTION

### Field of the Invention

The present invention relates to a method for eliminating selected microorganisms of one or several types within the human body and, more particularly, to a combined method for eliminating microorganisms within the human body that requires a mechanical pre-damage of the pathogens capsule, secondly intake of a safe dose of an external oxidant, plus parallel supplements that invoke controlled eNOS-expression (ROS/RNS) for a determined time, as a result producing internal radical oxidation/nitric species in the body. During eNOS-expression, the uncontrolled NO production from iNOS-expression causing fever, inflammation, formation of thrombocyte aggregation (thrombus, oedems) is brought back under control and aggregations are dissolved, sepsis is stopped. Thirdly the end of the oxidation period is intentionally invoked using antioxidants and provoking TH2 shifting, supporting the production of antibodies. Lastly, fourthly providing measures, strengthening the immune system with the supplementation of measured deficiencies of amino acids, vitamins, minerals, trace-elements, co-enzyms etc.

### Description of the Related Art

Several designs for a method for eliminating microorganisms within the human body have been designed in the past. None of them, however, include a method for eliminating microorganisms and their induced organism which selectively kills microorganisms in the human body in such an effective, fast, and soft manner. The method includes a first step which determinates the frequencies of vibrations at resonance of pathogens within a human body. The pathogens are then applied a slow cycling resonant frequency band to mechanically and evenly pre-damage the hulls of the different sized and formed family members of the same microorganism type.

The method further includes a second step including penetrating the damaged hulls of the pathogens with a safe dose of an external and parallel internal produced oxidation species to eliminate the microorganism. The external oxidation product may include chlorine dioxide in a watery solution (at a dose generally recognized as safe). The internally oxidation product is produced inside of the body as ROS (radical oxygen/oxide species), and/or RNS (radical nitrogen/nitric species), this results in a shifting of uncontrolled NO gas production from iNOS (induced Nitric Oxide Synthase) -expression towards controlled NO gas production from eNOS (endothelial Nitric Oxide Synthase) -expression. eNOS-expression is capable to do the oxidation effect on the damaged hulls of the damaged microorganisms.

This new method uses the intentional invoked eNOS-expression (using Vitamins B2, B3, B9, Zn Zinc, Fe Iron, and amino acids L-Arginine and L-Citruline) as a tool to reduce the free radicals in the body such as the dangerous Superoxide O—, Peroxynitrite ONOO—, Dinitro-trioxide N2O3 radicals, which would cause damage to the human cells if uncontrolled NO (Nitric Oxide) production is invoked from macrophages, or neutrophilic granulocytes or inflammatory interleukines causing iNOS-expression with 1000 times the NO production as in eNOS-expression.

In order to start eNOS-expression, the vitamins B2 (Riboflavine), B3 (Niacine), B9 (Folic Acid) [actually a derivate from vitamin B9 which is called BH4 “Tetrahydrobiopterin” and is formed from vitamin B9 inside of the body] are required for the donation of electrons in this biochemical process, it forms BH3+ and is recycled to BH4. Zinc forms with a Hem (iron) group a so called “dimere” which is required for eNOS-expression. Oxygen as O2 will react in this systems to donate Oxygen, forming NO gas and the B-vitamins electrons e- which are absorbed from receptors of the L-Arginine and forming NO Nictric Oxide as a gas and L-Citruline aminoacid as the reaction products. The NO as a gas is the internal oxidation product we used. During eNOS-expression the L-Citruline is recycled to L-Arginine. Several circumstances can lead to a so called eNOS-“uncoupling”, which leads to the production of superoxide O— instead of NO, which reacts with the existing NO gas to form peroxinitrite ONOO—, which is capable to irreversibly react with the folic acid derivate BH4 and oxidizes it to BH2 which cannot further be used for the eNOS-expression and the uncoupling occurs. This caused uncoupling leads to iNOS expression which produces 1000 times the quantity of NO gas and leads to the hyperproduction of the radicals superoxide O—, peroxinitrite ONOO— and dinitro-trioxide N2O3, which cause irreversible damage to cells, fever, formation of thrombus, edemas, inflammation and sepsis, specially with respect to Covid-19.

The method further includes a third step terminating and reducing the two different oxidation processes by providing antioxidants in form of Vitamin C and Vitamin E to neutralize the external oxidant CLO2 within 20 minutes. Parallel we are provoking a shifting towards TH2 (Thymus Helper Cell type 2) of the immune system supplementing Omega 3 fishoil, Vitamin K and D3. TH2 is the antagonist for ROS/RNS production or TH1 dominance. So if the shifting towards TH2 is initiated the production of the internal NO gas using eNOS-expression or ROS/RNS radical species is reduced, stopping the internal oxidation process and activating the TH2 of the immune system, leading to the increased production of antibodies and the application of B-cells of the immune systems defense.

A fourth step includes determining deficiencies within the body using a NLS system or laboratory blood analysis and supplementing the missing elements selectively according to the deficiencies encountered, thereby strengthening the immune system by providing the missing educts for biochemical reactions, such supplements as essential amino acids in an aqueous solutions as well as, vitamins, minerals, trace-elements and co-enzymes etc. to the human body.

It is known that there is a plurality of diseases caused by microorganisms and pathogens in medical practice. It is also known that antibiotics work only on bacteria and chemically damage or block the cell hull or capsules containing murine. Unfortunately, there is a large number of viruses and other pathogens where antibiotics do not work or where bacterial strains have already developed resistance to existing antibiotics. Additionally, many antibiotics normally do not eliminate only a specific type of pathogen. Good bacteria (of the intestinal flora for example) may also be eliminated during antibiotic treatment which may lead to further infections (molds for example) and diseases in the future. Therefore, there is a need to selectively eliminate pathogens, not damaging the good ones. This is a crucial advantage of the invention.

Applicant believes that a related reference corresponds to U.S. Pat. No. 7,165,451 issued for a detection of inorganic acid and biologic structures using resonant acoustic and/or resonant acoustic-EM energy. Applicant believes that another related reference corresponds to U.S. Pat. No. 5,658,322 issued for a system and method for generating bio-active frequencies. The generated frequencies are used to advantage in health science applications such as killing microorganisms and viruses and enhancing tissue generation.

However, the cited references differ from the present invention because they fail to evenly destroy different sizes of the same present pathogen type, to disclose the combination of using resonant frequency treatment to pre-damage the hull of a pathogen and then introducing an oxidation step to eliminate a specific type of pathogen. None of them is capable to bring inflammations, thrombus, edemas caused from iNOS-expression under control, with its dramatic damaging effects inside of the body, nor do they explicitly strengthen the immune system to take over control again after the treatment. Advantageously, the viruses are oxidized in a way so they cannot multiply anymore.

Other documents describing the closest subject matter provide for a number of more or less complicated features that fail to solve the problem in an efficient and economical way. None of these patents suggest the novel features of the present invention.

### SUMMARY OF THE INVENTION

It is one of the objects of the present invention to provide a method for eliminating specific microorganisms which allows a medical practitioner to selectively target a pathogen type to be eliminated, leaving the good microorganisms untouched. At the same time allowing to taking control over the inflammations, aggregation of thrombocytes leading to thrombus, edemas and sepsis, caused by a NO poisoning or iNOS-expression with the high liberation of peroxinitrite ONOO—, superoxide O— and dinitro-trioxide N2O3.

It is another object of this invention to provide a method eliminating microorganisms, which eliminates pathogens of the same population but varies in, sizes, lengths, diameters and age, efficiently and faster than other methods and with less side effects.

It is still another object of the present invention to provide a method for eliminating microorganisms, that allows to selectively destroy pathogens while leaving non-threatening microorganisms untouched. That is done independent of apparatus producers or producers of the chemical watery solution of ClO2 or supplements like essential amino acids, vitamins, minerals, trace-elements and co-enzyms etc.

It is yet another object of this invention to provide such a method that is inexpensive to implement and maintain while retaining its effectiveness.

It is yet another object of this invention to provide a synergetic effect that only the combination of the described and undertaken steps allow. This is specially the combination of the mechanical fatigue using vibrations and the oxidation process.

It is still another object of this invention, that the invoked eNOS expression using L-Citruline will lead to the possibility to take over control caused by an eNOS-uncoupling leading to iNOS-expression, hyper inflammation, aggregation of thrombocytes leading to thrombus and edemas and sepsis, caused by a 1000 times production of NO, superoxide O—, peroxinitrite ONOO— and dinitro-trioxide N2O3.

A further object of this invention is the intentional stop of the oxidation process and invoking TH2 shifting leading to the increased production of antibodies of the immune system and B-cells to fight the pathogens.

Further objects of the invention will be brought out in the following part of the specification, wherein detailed description is for the purpose of fully disclosing the invention without placing limitations thereon.

### BRIEF DESCRIPTION OF THE DRAWINGS

With the above and other related objects in view, the invention consists in the details of construction and combination of parts as will be more fully understood from the following description, when read in conjunction with the accompanying drawings in which:

FIG. 1 represents a flow chart for a method 10 including a first step 20, a second step 40, a third step 60 and a fourth step 80 in accordance to an embodiment of the present invention.

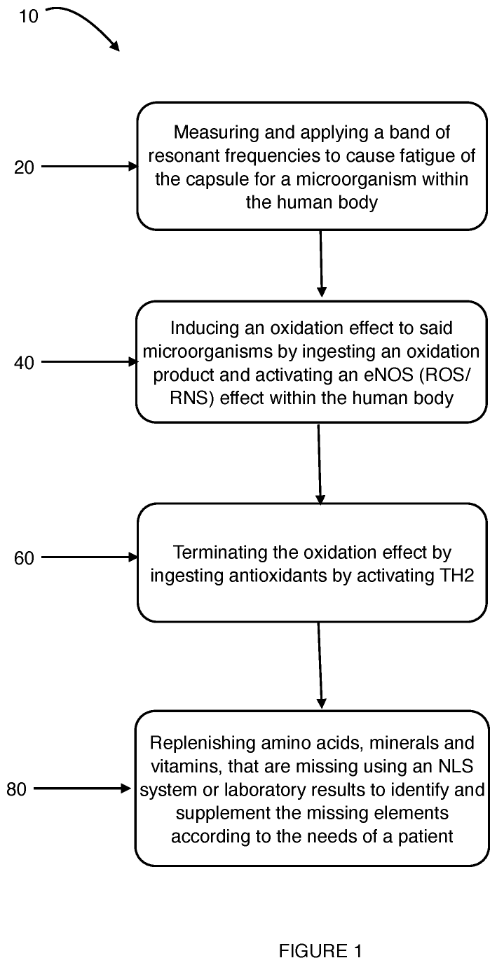

### DETAILED DESCRIPTION OF THE EMBODIMENTS OF THE INVENTION

Referring now to the drawings, where the present invention is generally referred to with numeral 10, it can be observed a method 10 for eliminating selectively microorganisms which basically include a first step 20, a second step 40, a third step 60, and a fourth step 80.

First step 20 includes measuring frequencies of pathogens and applying a varying resonance frequency to the pathogens according to existing different sizes etc. This step is accomplished through an analytic electronic method with electrodes. The electrodes emit DC current induced at discrete frequencies by stepping up in discrete selected steps within a frequency band, offering vibration energy to the pathogens. If resonance is offered and achieved for a certain pathogen it is able to start to vibrate using this offered energy at a maximum amplitude. It is then measured the frequency that results resonance and how much energy is absorbed from these pathogens that start to vibrate at this specific resonance frequency. This physical effect is known as prior art in the field of resonant frequencies and pathogens. Every microorganism of a specific type and size has a discrete resonance frequency and will vibrate in its first harmonic resonance frequency form at maximum amplitude, in the present method, if an electromagnetic wave or current is applied at this resonance frequency. The offered vibrations energy which is applied to provide a certain level of intensity is partially absorbed by the microorganism and transformed into heat and further into mechanical fatigue in the hull/capsule. If the vibrations are applied long enough the capsule of the microorganism will even rupture. This damage effect is known as “fatigue”.

Pathogens of a specific type are always of varying size, length, diameter and consistency, resulting in a different mass of the pathogens and/or stiffness of the capsule. As a result, every pathogen will vibrate at a maximum amplitude and maximum intensity at a slightly different resonance frequency (This frequency omega=SQRT(c/m), c: stiffness, in: mass). If only a static frequency is applied, not all the pathogens of different mass and stiffness will vibrate with the same intensity at resonant frequency at the provided frequency and with the same amplitude of hull movement, they will not all receive the same fatigue, because the resonance frequency depends on the square root of (stiffness divided by its mass) of the pathogen. As a result, not all pathogens die or get the same level of damage/crack/weakness. A variable resonant frequency band should therefore be applied to the pathogens to evenly fatigue all of the pathogens. This effect can be applied to the pathogens until the microorganism eventually explode and everything within the microorganism is released and the microorganisms will eventually die. The present invention utilizes this fatigue effect; however, the fatigue effect is only applied to produce a small defect in the hull in form of a pre-damage/crack/weakness, which is sufficient if combined with the second oxidation step to destroy the pathogen. The fatigue effect is not used to completely destroy the microorganisms hull or capsule at this step.

Human cells are known to have a resonant frequency around eight million Hertz and microorganisms and pathogens are known to have a resonant frequency mostly below 2 million Hertz. As a result, no damage is done to human cells during the process of applying the resonant frequencies to the microorganisms and pathogens.

For example, if we measure a frequency of 200,000 Hertz using discrete steps of 1000 Hertz for detecting the resonance frequency of a microorganism. It is known that the human body may contain a large number of different microorganisms therein and that in order for a disease to develop symptoms or illness a certain threshold of microorganisms need to be exceeded. The more pathogens of the same type that are present, the more energy is absorbed during the detection step—so speaking, a peak of absorption of energy indicates the presence of a big amount of the same microorganism type even though the immune system is fighting to control it. The threshold depends on the type of microorganism and symptoms/illness level. Using the measured frequency in combination with a polymerase chain reaction, a relation is made as to the resonant frequency of the microorganisms and the illness/symptoms. Afterwards a standard frequency generator is used to generate a frequency band which is half of the discrete step width (here it was 1000 Hz steps), so from +−500 Hertz around the measured absorption frequency. Using different step widths require adopted bands of plus and minus ½ the step width. The smaller the discrete steps, the more precise and smaller are the selected frequency bands, leading to a more efficient pre-damage of the hull in the same time, more cycles during the same time. In this example, the frequency generator begins at 199,500 Hertz and steps up to 200,500 Hertz, by stepping up every second by 1 Hertz. One complete stepping up and down is called a cycle. The frequency generator is attached to an antenna or contact electrodes which apply a maximum of 15 VDC output. The antenna or contact electrodes are applied to a patient and the process is repeated several times/cycles to produce small defects/cracks/weaknesses in the hulls of microorganisms, but sufficiently profound. This process takes considerably less time than attempting to completely rupture all the different sized cells of the microorganisms using only this process.

Second step 40 includes penetrating the damaged hulls of the microorganism with a low dose of an ingested external oxidation product and an oxidation product generated inside the body as an eNOS-expression (ROS/RNS). Normally disinfection products need a high dose in order to destroy the hull of a microorganism by their sole use. Additionally, these disinfection products are aggressive and may cause injuries if swallowed at these concentrations. However, the present method provides a solution to the problems involved in the use of disinfection products. The present invention applies a very low dose of an external ingested disinfection product plus, in parallel invokes internal oxidation/nitric species being produced, known as eNOS-expression, towards the fatigued hulls of the microorganisms and thereby selectively destroys them preferably easier due to the crack or damage or local weakness in the capsule. In a virus for example the double lipid membrane of the microorganisms is more easily penetrated due to their fatigued/weakened nature and allows for oxidation to take place for the guanine of the ARN (ribo nuclein acid) or ADN (desoxiribo nuclein acid) of the microorganism.

It is known that the microorganisms of a virus do not have a cellular core nor a metabolism of its own. The viral microorganisms contain ADN or ARN within the cellular hull which need to invade a human cell in order to reproduce. Additionally, both the ADN and ARN of the microorganisms include a guanine base. The present invention utilizes external ingestion of chlorine dioxide (CIO2) in a watery solution and eNOS-expression to change the guanine base into a guanine-oxidized base. As a result of the oxidation, the code of the ARN that is built of a series of bases (3 bases=triplet or codon, form an amino acid) is interrupted because the guanine is replaced by its oxide thereby preventing the virus from multiplying again, the bio-molecular transcription process is interrupted. The present invention introduces chlorine dioxide (CIO2) in an aqueous solution of 0.75 ppm (which is below 0.8 ppm, which is generally recognized as safe by the FDA). 5 drops of 3000-ppm chlorine dioxide (CIO2) are given to 1 liter of water resulting in 0.75 ppm drinkable solution. 1 ccm of 3000 ppm equals 20 drops or 3 mg, so 5 drops give a 0.75 mg/ltr or 0.75 ppm concentration.

The selection of this product will not affect healthy human cells as this ClO2 carries an oxidation potential that is lower than an oxidation potential of the healthy human cells, apart as it is applied at this low safe dose. The fatigued hulls of the microorganisms that results from first step 20 allows the product together with eNOS expression (ROS/RNS) to infiltrate the microorganisms and begin oxidation. eNOS (endothelial nitric oxide synthase). ClO2 has a stronger oxidation reaction if the reaction partner is acidious, so the lower the pH value (pH<7.0 is an acid), the stronger the reaction of ClO2. For this reason it is a selective oxidant. Only pathogens have a pH<7.0 good germs do not. Blood for example has a pH of about 7.35 . . . 7.45.

In various implementations, first step 20, second step 40, a third step 60 and a fourth step 80 may be used in different cases to eliminate SARS-CoV 2 and control Covid-19.

During the process of the first step 20 the antenna or electrodes cycle the measured and programmed resonance frequencies by stepping up and stepping down several cycles that are applied to the human body, the external oxidation is done in a way of drinking every 30 minutes a glass of water containing 0.75 ppm ClO2 in watery solution. In parallel, the eNOS expression is started at the same time, by supplementing the following listed products at the same time, this is ingested together with the CLO2 in watery solution:

### Initially:

2 grains of L-Citruline-Malate,
1 gram total of L-Arginine-Malate, L-Arginine HCL

### 100 mg of Vitamin B2 (Riboflavine),

### 100 mg of Vitamin B3 (NAD, Niacine),

0.8 mg of Vitamin B9 (Folic acid) or 5-MTHF (5-Methyl Tetra Hydro Folate),
25 mg of Zinc gluconate, and 25 mg Iron citrate. (25 mg based on Zn and Fe)
Thereafter every hour for keeping the eNOS-expression alive:
1 gram of L-Citruline-Malate
0.5 gram of L-Arginine-Malate, L-Arginine HCL

### 10 mg of Vitamin B2 (Riboflavine),

### 10 mg of Vitamin B3 (NAD, Niacine),

0.1 mg of Vitamin B9 (Folic acid) or 5-MTHF (5-Methyl Tetra Hydro Folate),
2.5 mg of Zinc gluconate, and 2.5 mg Iron citrate. (2.5 mg based on Zn and Fe)

These values are based on the weight of a person with 80 kg weight. The amino acids are dissolved in a glass of water or swallowed in capsules as the rest of the vitamins and minerals and a glass of water is ingested together at the same time, that contains the CLO2 in 0.75 ppm.

The supplementation of these above products prevents the eNOS uncoupling and if it was uncoupled at the begin, to restart eNOS-expression, resulting in downgrading of uncontrolled iNOS-expression which caused inflammation and fever and the building of thrombus as an agglomeration of blood thrombocytes etc. The eNOS-expression is able to also support to dissolve these aggregations independent of supplemental medication. L-Citruline-Malate (amino acid), a product from eNOS-expression bio-chemical reaction (besides the NO), reduces iNOS and brings NO hyperexpression and the high radical production of superoxide O—, peroxinitrite ONOO— and dinitro-trioxide N2O3 back under control.

Method 10 further includes a third step 60 which involves terminating the oxidation process, which was initiated from second step 40 by a way of introducing antioxidants using Vitamin E (800 i.U), Vitamin C (500 mg), to stop the oxidation of the CLO2 external oxidation product.

500 mg Glutathion amino acid, MnSOD in form of 15 mg Manganese Mn gluconate (15 mg based on the metal Mn), and 15 mg ZnSOD in form of Zinc Zn gluconate (15 mg based on the metal Zn) into the human body and TH2 (Thymus Helper Cells type 2) shifting, ingesting 2000 mg Omega 3 fishoil, 2.5 mg Vitamin K (K1+K2) together with 10.000 i.U. Vitamin D3 based on the weight of an 80 kg person.

The antioxidants should not be taken during the day or before the treatment, the same is true for the products to provoke TH2 shifting, the oxidation effect resulting from second step 40 will not occur since the antioxidant and TH2 shifting will neutralize the external and internal oxidation products. As a result, method 10 includes inducing the oxidation effect during the day and terminating the oxidation effect latest at night, half an hour before the patient goes to bed.

In a given treatment, steps 20 and 40 are applied. As soon as the symptoms of Covid-19 reduce, the ingestion of external ClO2 in watery solution and the eNOS expression is still kept up for another 2 hours, as described above. If the treatment was intended to be done and the patient did not show any symptoms but was positively tested on SARS-CoV2, then step 20 and 40 is sustained for max. 3 hours as a preventive measure to show the effect of eliminating the SARS-CoV2 and preventing Covid-19. Afterwards, after another hour after the last intake of the oxidation products, step 60 is activated using antioxidants and TH2 shifting. The products that work for step 60 are a combination of Vitamin E, Vitamin C, Glutathion amino acid, MnSOD (Manganese Super Oxide Dismutase, supplementing the metal manganese, MnSOD is produced in the body) in form of Mn gluconate, and ZnSOD (Zinc Super Oxide Dismutase, supplementing the zinc, ZnSOD is produced in the body) in form of Zn gluconate. The doses are as explained above, are considered for a 80 kg person.

Patients of Covid-19 and SARS-CoV2 then have their immune system strengthened by means of step 80, directly 20 minutes after ingesting the supplements from Step 60. During the treatment process the existing levels of amino acids, vitamins, minerals, trace-elements and co-enzymes are measured and supplemented according to those measured to be lacking with adopted doses. This can be done from a blood analysis or adequate systems. It is important to detect if there are any missing levels and supplement only accordingly, physical exercise and sports also help during this replenishing step because they influence the absorption rate. It makes no sense to overfeed in means of “the more the better”, only the missing educts should be supplemented to avoid reverse side effects. It is important to understand, that vitamins, minerals, aminoacids, co-enzyms etc. contribute to the biochemical reactions of the immune system and enable it to regain strength. The exact dose is matter to the theurapeuts considerations and experiences.

Part of the immune system (in TH2) develops and uses antibodies to further destroy intruded pathogens. Antibodies are proteins, which are synthesized in the lymph cells using amino acids. The 20 amino acids used contain 8 essential and 12 non-essential amino acids. The 12 non-essential amino acids can be synthesized inside the body if sufficient essential amino acids, minerals, vitamins, co-enzymes trace-elements and co-factors are available. A low protein diet can lead to these amino acid deficiencies. Vegan or vegetarian diets lead to a depletion of B-Vitamins etc. Doing no exercise or no sports leads to a low level of antioxidants leading to eNOS uncoupling due to the lack of free radical neutralization. Parasites might lead to depletion of available metals (iron, hemoglobin, and zinc etc) and vitamins/minerals etc. in the body for bio-chemical reactions and might lead to the blockage of available L-Arginine to prevent their own destruction from eNOS expression (using elevated Arginase release). A lack of L-Arginine causes eNOS-uncoupling and iNOS-expression as well as the production of dangerous Superoxide and Peroxinitrite and Dinitro-Trioxide.

The amino acids, vitamins, minerals, etc. are also often reduced from microorganisms (parasites) which invade the body. For example, evolutionary the Toxoplasmosis germ induces Arginase (an enzyme=protein) that reduces the available L-Arginine and causes uncoupling of eNOS, in order to protect itself from destruction.

A similar effect of eNOS uncoupling is detected at elevated levels of ADMA (asymmetric di methyl arginine) which appears on clinical pictures such as kidney deficiencies, high blood pressure, diabetics, high triglycerides and others (factors up to 12 times to normal ADMA levels are present) that block the electron receptors on the L-Arginine. This leads to the paradox situation, that even though the L-Arginine levels are normal, still eNOS uncoupling occurs, because the L-Arginine is blocked. This is one of the reasons, why older people are more likely to develop a more severe form of Covid-19 and form part of the high-risk groups, because they have elevated ADMA levels apart from other influences resulting in iNOS-expression. The supplementation of L-Arginine and L-Citruline together is able to break this paradox and together with the explained supplementation of B-Vitamins and metals in form of organic minerals start the eNOS-expression again.

Fourth step 80 ends method 10.

Method 10 may be implemented for various applications and is not limited to SARS-CoV 2, Corona Virus. Other applications include using method 10 on cases of Herpes, Dengue, Malaria and other known microorganisms, in special antibiotic resistant or even multi-resistant pathogens or even in case of mutations where successful vaccines are not effective anymore. This is especially important in respect to Covid-19, because vaccines only protect to a certain point and with the present method people that get infected after the vaccination can be detoxified from SARS-Cov2. New mutations of the virus can also easily be eliminated, adopting if necessary the frequency band. The virus has no chance to escape the mechanical fatigue and the chemical oxidation process.

The following list provides some examples which is not exhaustive of which types of pathogens can be eliminated this way. For example, Klebsiella pneumoniae, Staphylococcus aureus, Escheria coli, EHEC etc. Also, unknown pathogens (for example the pathogens causing MERS-CoV etc.) can be detected and eliminated with this combined method, the treatment only needs to trace the frequencies of the pathogens participating, regardless of their name or origin. In case of MERS-CoV it was discovered that several microorganism work together to cause the outbreak of the illness. Tropical illnesses where the pathogen is known, but there is no medication available such as Ebola, Schistosomiasis, Leishmaniosis and others etc. can be successfully treated with this method. Method 10 allows a user to target any specific microorganism and eliminate them using the oxidation process, together with the resonance frequencies and combined oxidation steps, the intentional stop of the oxidation process and then activating of TH2 shifting (and the production of antibodies and B-cells) and finally the strengthening of the immune system supplementing the missing educts.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

What is claimed is:

1. A method for eliminating specific microorganisms, comprising:
a) measuring and detecting resonant frequencies of microorganisms within a human body and applying a band of resonant frequencies to said human body through a frequency generator and antenna or contact electrodes to cause a damage, crack, or weakening of a capsule of microorganisms within the human body;
b) inducing an oxidation effect to said microorganisms by ingesting an aqueous mixture of less than 0.8 ppm of Chlorine Dioxide, and simultaneously internally provoking an eNOS-expression and an internal production of NO gas over a prolonged period together with the application of the band of resonant frequencies;
c) reducing an iNOS-expression and accompanied effects of fever, inflammation, aggregation of thrombocytes leading to thrombus and edemas, sepsis caused by NO-poisening, superoxide, peroxinitrite and dinitro-trioxide radicals;
d) terminating said oxidation effect by ingesting antioxidant supplements including Vitamin E and Vitamin C to terminate CLO2 external oxidation, said terminating of said oxidation effect further including ingesting Glutathion (Amino acid), MnSOD in form of Mn gluconate, and ZnSOD in form of Zn gluconate, and provoking a TH2 shift by ingesting Omega 3 fishoil, Vitamin K plus Vitamin D3; to control internal oxidation from ROS/RNS during TH1 and the eNOS-expression; and
e) replenishing deficient amino acids in the human body with an aqueous solution of vitamins, minerals, trace-elements and co-enzymes by ingesting them.

2. The method for eliminating microorganisms of claim 1 wherein said band of resonant frequencies is plus ½ step width up and minus ½ step width down of a measured absorption frequency to form a frequency band width of the cycle.

3. The method for eliminating microorganisms of claim 1 wherein said antioxidant supplements being ingested are provided in liquid, capsule, powder or tablet form, including any combination thereof.

4. The method for eliminating microorganisms of claim 1, wherein the reduction of the uncontrolled iNOS-expression which causes fever, inflammation, and the aggregation of thrombocytes forming thrombus and edemas, is achieved by means of inducing the eNOS-expression and L-Citruline.

5. The method for eliminating microorganisms of claim 1 wherein said essential amino acids, vitamins, minerals, trace-elements and co-enzymes are adapted to refill depleted deposits within said human body which are determined according to a measurement from a laboratory blood analysis or an NLS system.

6. The method for eliminating microorganisms of claim 1 wherein said aqueous mixture contains at most 5 drops of 3000 parts per million of chlorine dioxide per liter of water resulting in 0.75 ppm ClO2.

7. The method for eliminating microorganisms of claim 1 wherein the terminating of the oxidation effect occurs at night, at least half an hour before entering a sleeping state.

8. A method for eliminating specific microorganisms, consisting of:
a) measuring and detecting resonant frequencies of microorganisms within a human body and applying a band of resonant frequencies to said human body through a frequency generator and antenna or contact electrodes to cause a damage, crack, or weakening of a capsule of microorganisms within the human body;
b) inducing an oxidation effect to said microorganisms by ingesting an aqueous mixture containing at most 5 drops of 3000 parts per million of chlorine dioxide per liter of water resulting in 0.75 ppm ClO2 ingested every 30 minutes, and simultaneously internally provoking an eNOS-expression and an internal production of NO gas over a prolonged period together with the application of the band of resonant frequencies;
c) reducing an iNOS-expression and accompanied effects of fever, inflammation, aggregation of thrombocytes leading to thrombus and edemas, sepsis caused by NO-poisening, superoxide, peroxinitrite and dinitro-trioxide radicals;
d) terminating said oxidation effect by ingesting antioxidant supplements including Vitamin E and Vitamin C to terminate CLO2 external oxidation, said terminating of said oxidation effect further including ingesting Glutathion (Amino acid), MnSOD in form of Mn gluconate, and ZnSOD in form of Zn gluconate, and provoking a TH2 shift by ingesting Omega 3 fishoil, Vitamin K plus Vitamin D3; to control internal oxidation from ROS/RNS during TH1 and the eNOS-expression; and
e) replenishing deficient amino acids in the human body with an aqueous solution of vitamins, minerals, trace-elements and co-enzymes by ingesting them.
